(19) United States
(12) Patent Application Publication
(10) Pub. No.: US 2005/0065595 A1
(43) Pub. Date: Mar. 24, 2005

(54) Implants containing combretastatin a-4

(76) Inventors: David Chaplin, Watlington (GB); Stephan Wnendt, Aachen (DE); Bernd Kuttler, Reutlingen (DE); Gunter Lorenz, Tubingen (DE)

Correspondence Address: James Velema;IP Department Oxigene, 321 Arsenal Street, Watertown, MA, 02472, US

(21) Appl. No.: 10/488,515

(22) PCT Filed: Sep. 3, 2002
(86) PCT No.: PCT/EP02/09836

(30) Foreign Application Priority Data
Sep. 3, 2001 (DE) 101 42 881.2
Sep. 3, 2001 (DE) 101 42 897.9

(51) Int. Cl. A61F 2/06; A61F 2/00

(52) U.S. Cl. 623/1.42; 623/1.44; 424/426; 427/2.25

(57) ABSTRACT

The subject matter of this invention relates to implants, in particular intracavernous or intravascular implants, preferably for use in the treatment or prophylaxis of coronary or peripheral vascular occlusions or vascular narrowings or stenoses, in particular for use in the prophylaxis of a restenosis, which implants contain CA4 or CA4P in a chemically covalently or noncovalently bonded or a physically fixed form, and methods for the production and application thereof.

The subject matter of this invention relates to implants, in particular intracavernous or intravascular implants, preferably for use in the treatment or prophylaxis of coronary or peripheral vascular occlusions or vascular narrowings or stenoses, in particular for use in the prophylaxis of a restenosis, which implants contain combretastatin A-4 (CA4) or combretastatin A-4 phosphate (CA4P) in a chemically covalently or noncovalently bonded or a physically fixed form, and methods for the production and application thereof.

The formation of arteriosclerotic lesions in the arterial blood vessels is an underlying disease with a large spectrum of clinical symptoms which range from angina pectoris and claudicatio intermittens to myocardial infarction and ischemic stroke, all due to atheromatous formation and/or stenotic lesions. The term stenotic lesions defines the local reduction of the vascular lumen to less than 60-70% of its normal diameter, which in turn leads to a markedly reduced supply of oxygen and nutrients to the tissue involved. In spite of the fact that in the past decade, drug therapy (statins, ACE inhibitors, gpIIa/IIIb blockers and plasminogen activators) has led to satisfactory therapeutic results, in particular in the area of cardiovascular diseases, many patients who have developed a complete ischemic syndrome still require surgical interventions (bypass operations, etc.). In addition, these operations are relatively complicated and expensive and also pose the risk of serious complications.

To prevent ischemic coronary diseases, minimally invasive surgical procedures have been developed. The invention of percutaneous transluminal coronary angioplasty (PTCA) in the late 1970s was a tremendous breakthrough in cardiology. In PTCA, inflatable balloons are used which are pushed up to the stenotic lesion in the coronary arteries. These balloons are subsequently inflated at the targeted sites and cause the stenotic region to be dilated. A similar procedure can also be used to dilate carotid or peripheral arteries.

Very early on, however, it was discovered that in a relatively large number of PTCA patients, stenoses recurred in the regions which had been dilated with the balloon catheter. In the course of this discovery, it was found that this so-called restenosis is caused by a neoformation of the vascular architecture of tissue layers. The introduction of tubular vascular metal implants, so-called stents, in the transluminal treatment of stenotic regions improved the situation dramatically. In clinical studies (Serruys et al., N. Engl. J. Med. 331 (1994, pp. 489-495), it was demonstrated that the use of stents at the sites which had been dilated with balloons was able to reduce the recurrence of stenoses from approximately 45% to approximately 30%. Although this definitely represents a significant improvement in the prevention of restenoses, there is still considerable room for therapeutic improvements.

In the course of detailed studies of the pathophysiology of restenoses in the stent, it was discovered that this particular restenosis differs from the PTCA-induced restenosis. Inflammatory reactions, hyperproliferation, and the proliferation of smooth muscle cells (SMCs) are important factors of the neointimal formation which leads to a restenosis in the stent. In the animal restenosis model and even in human tissue, it was found that the hyperproliferation of the SMCs goes hand in hand with an infiltration of the tissues around the bracing structures of the stent by macrophages and T cells (Grewe et al., J. Am. Coll. Cardiol. 35 (2000), pp. 157-163). By analogy to other clinical indications in which inflammatory reactions and the hyperproliferation of cells play a role and which can be controlled by drug therapy, an attempt was made to treat the restenosis by means of drugs. Selected active substances were administered orally or intravenously or were delivered to the site of action by means of perforated catheters. Unfortunately, none of the active substances used to date has so far been able to markedly reduce a restenosis (Gruberg et al., Exp. Opin. Invest. Drugs 9 (2000) pp. 2555-2578).

The direct administration of pharmacologically active substances by means of stents that are coated with the active substance appears to be the method of choice. Animal experiments and preliminary results of clinical tests with stents coated with active substances indicate that a delayed release of immunosuppressive or antiproliferative active substances may reduce the risk of a restenosis. Paclitaxel, a cytostatic drug, and rapamycin, an immunosuppressive drug, were tested in animal experiments. Both compounds inhibit the neointimal development (Herdeg et al., Semin. Interven. Cardiol. 3 (1998), pp. 197-199; Hunter et al., Adv. Drug Delivery Rev. 26 (1997), pp. 199-207; Burke et al., J. Cardiovasc. Pharmacol. 33 (1999), pp. 829-835; Gallo et al., Circulation 99 (1999), pp. 2164-2170). Yet, 6 months after the implantation of stents coated with paclitaxel in swine, it was found that the effect had been neutralized (Heldman, International Local Drug Delivery meeting and Cardiovascular Course on Radiation, Geneva, Jan. 25-27, 2001). The efficacy of rapamycin was excellent, and in the first clinical applications, it was found that the restenosis had disappeared completely (Sousa et al., Circulation 103 (2001), pp. 192-195). On the other hand, this seems to go hand in hand with a delayed healing of the vascular wall that had been injured in the course of balloon angioplasty and stent placement.

Generally speaking, a balance between healing the arterial wall following angioplasty and stent placement, on the one hand, and inhibiting the neointimal development, on the other hand, is very important. To achieve this balance, the goal should be the use of active substances which interfere selectively with specific mechanisms that lead to a neointimal development.

Thus, the problem to be solved by the present invention was to make available implants with properties favorable for treating and preventing a restenosis.

Therefore, the subject matter of the present invention is an implant containing CA4 or CA4P in a chemically covalently or noncovalently bonded or a physically fixed form and, optionally, a minimum of one additional active substance.

It should be noted that the term active substance, including the active substance CA4 or CA4P, mentioned in the context of the present invention also refers to any direct derivative of the active substance as well as to all types of salts, enantiomers, racemates, bases, or free acids of the active substance and to mixtures thereof.

Preferably, the implant is an intracavernous, preferably an intravascular implant.

In this context, intracavernous refers to any hollow space, in particular any hollow organ or hollow organs, such as blood vessels, esophagi, ureters, bile ducts, etc.

Intravascular refers in particular to the use in a blood vessel.

In addition, it is to be preferred that the implant is suitable for the treatment or prophylaxis of vascular narrowings or stenoses, preferably for the prophylaxis of restenoses, in particular of coronary or peripheral vascular occlusions.

Therefore, an intracavernous, preferably an intravascular implant for the treatment or prophylaxis of vascular narrowings or stenoses, preferably for the prophylaxis of restenoses, in particular of coronary or peripheral vascular occlusion, containing CA4 or CA4P in a chemically covalently or noncovalently bonded or a physically fixed form and, optionally, a minimum of one additional active substance.

The active substance combretastatin A-4 (2-methoxy-5-[(1Z)-2-(3,4,5-trimethoxyphenyl)ethenyl]phenol) is a new drug for the treatment of tumors which inhibits the accumulation of microtubuli during cell division (El-Zayat et al., Anti-Cancer Drugs 4 (1993), pp. 19-25; Dorr et al., Invest. New Drugs 14 (1996), pp. 131-137). Cell culture tests demonstrated that the water-soluble derivative of CA4, i.e., CA4P (disodium combretastatin-A4-3-O-phosphate), inhibits the growth of many different tumor cell lines (El-Zayat et al., Anti-Cancer Drugs 4 (1993), pp. 19-25). In vivo, CA4P is very rapidly transformed into CA4 by alkaline phosphatases. The IC₅₀ values which were determined in the cell culture studies were in a range of 0.2-6 nM. In animal experiments, CA4P was found to markedly inhibit the growth of tumors in various animal models (e.g., Landuyt et al., Eur. J. Cancer 36 (2000), pp. 1833-1843); based on the knowledge available so far, this effect is attributable to an inhibition of the blood circulation in the tumor tissue (Dark et al., Cancer Res. 57 (1997), pp. 1829-1834; Horsman et al., Int. J. Rad. Oncol. Biol. Phys. 42 (1988), pp. 895-898). This change in the blood circulation of the tumor tissue subsequently leads to an inhibition of the growth and to tissue death. The anti-tumor effect was observed at doses between 5 and 50 mg/kg/d.

Cell culture tests with CA4 on native cells of the smooth muscles (smooth muscle cells, SMCs) demonstrated a very considerable inhibition of the cell division which was reached at markedly lower concentrations that had been used with conventionally used anti-tumor drugs, such as paclitaxel (see annex 1). In these experiments, the IC₅₀ value of CA4 was approximately 0.5 nM, that of paclitaxel, on the other hand, was approximately 10 nM. Cell culture studies with endothelial cells indicate that following a treatment with CA4, the endothelial cells are able to recover more rapidly than the SMCs. This result is of considerable importance to the application for the prophylaxis of in-stent restenoses since the most important objective in this case is to not interfere with the growth of the endothelial cells, but to inhibit the growth of SMCs during cell division. Endothelial cells line the vascular walls and protect the vessels against the formation of clots and against hypertension. After conclusion of the healing process, the surface of the stent is covered by an endothelial layer.

In this context, stenosis is defined as the occlusion or narrowing of a vessel, and restenosis is defined as the recurrence of stenoses.

Furthermore, in this context, the term “containing” also includes, for example, a noncovalently bonded coating.

In this context, “peripheral” refers in particular to vessels and other hollow organs outside the heart and the coronary vessels.

“Chemically noncovalently bonded” refers in particular to bonds due to interactions, such as hydrogen bridges, hydrophobic interactions, van der Waals forces, etc.

“Physically fixed” refers to an inclusion, e.g., by means of a membrane inside a hole, or steric entrapment due to the choice of the size of the openings, etc.

An implant is any type of artificial object which is inserted (even if only for a limited time). It refers in particular to intracavernous, for example, intravascular, implants. Examples include stents, grafts, stent grafts, graft connectors, guide wires, catheter pumps, or catheters.

In the context of this invention, a stent is an implant with a hollow inside and with a minimum of two openings and, in most cases, with a circular or elliptical cross section but also with any other cross section (in most cases, made of metal but, optionally, also made of synthetic materials and polymers) which has an openwork, lattice-like structure and which is implanted into vessels, in particular into blood vessels, to keep said vessels patent and/or functioning.

In the context of this invention, a graft is an oblong implant with a hollow inside and with a minimum of two openings and, in most cases, with a circular or elliptical cross section but also with any other cross section, and with a minimum of one polymer surface that may be homogeneous or optionally woven from different strands and which is closed and impermeable to water and/or corpuscular blood components, which implant generally serves as a vascular prosthesis and which in most cases is used in injured vessels or in the place of vessels.

A stent graft in the context of this invention is defined as a combination of a stent and a graft. Accordingly, a stent graft is basically a vascular prosthesis reinforced by a stent (graft see above), with the polymer layer again being homogeneous or optionally woven from different strands and being closed and impermeable to water and/or corpuscular blood components. More specifically, a stent graft is a stent which, on a minimum of 20% of the surface of the implant, has an openwork (lattice-like) outer layer, preferably made of metal, and a minimum of one polymer layer which may be homogeneous or optionally woven from different strands and which is closed and impermeable to water and/or corpuscular blood components, which polymer layer may be located inside or outside this outer layer, and optionally (if a broken-work layer is present on the outside), an additional openwork (lattice-like) inner layer, preferably made of metal, inside the polymer layer and/or a polymer layer which may be homogeneous or optionally woven from different strands and which is closed and impermeable to water and/or corpuscular blood components.

In the context of this invention, a graft connector is defined as an implant which connects a minimum of two grafts and/or stent grafts and/or vessels with each other, which is made of the materials defined for the grafts or stent grafts and which has the structure defined for these [grafts or stent grafts] and which, accordingly, has a minimum of two, preferably 3 or 4, openings, and in particular an asymmetrical T shape.

In the context of this invention, a catheter is defined as a tubular instrument for introduction into hollow organs. In a narrower sense which is preferred in this context, catheters are guide or balloon catheters.

In the context of this invention, a catheter pump is a catheter that has a pump affixed to its tip.

According to another preferred embodiment of the implant according to the present invention, the implant has a minimum of one metal or metal alloy closed or openwork layer or surface which is homogeneous or formed from different strands.

In the context of this invention, metal or metal alloy refers especially to steel and steel alloys, but also to nickel and nickel alloys, with the term metal including any metal alloys.

Openwork structures are in particular lattice-like, woven or braided structures.

According to another preferred embodiment of the implant according to the present invention, the implant has a minimum of one closed or open-work layer or surface which consists of a polymer and which is homogeneous or formed from different strands.

In one preferred embodiment of the present invention, the implant has a minimum of one polymer layer which completely or partially covers a closed or open-work layer or surface which consists of a metal or a metal alloy and which is homogeneous or formed from different strands, preferably a structure consisting of a metal or a metal alloy and is optionally lattice-like.

According to an especially useful embodiment of the present invention, the implant has a minimum of one closed or open-work layer or surface which consists of a metal or a metal alloy and which is homogeneous or formed from different strands and a minimum of one closed or open-work layer or surface which consists of a polymer and which is homogeneous or formed from different strands.

In this context, it is especially useful if the metal or metal-alloy layer or surface of this implant is an optionally lattice-like structure made of metal or a metal alloy and/or the layer or surface made of a polymer is homogeneously closed or woven and/or impermeable to water and corpuscles and if the layers and surfaces from the outside to the inside are arranged in the following sequence: metal-polymer, polymer-metal, metal-polymer-metal, or polymer-metal-polymer and/or if either the layer or surface consisting of a polymer is not chemically (covalently or noncovalently) bonded to the layer or surface consisting of a metal or a metal alloy or if the layer of surface consisting of a polymer is bonded to the layer or surface consisting of a metal or a metal alloy by means of an adhesive.

It is also useful if the polymer used in the implants is Dacron, polytetrafluoroethylene (PTFE/Teflon®), expandable or unexpandable, or polyurethane; preferably polytetrafluoroethylene (PTFE), expandable or unexpandable; or polyurethane; in particular, PTFE.

According another preferred embodiment of the present invention, the implant is a stent, a stent graft, a graft, a graft connector, a guide wire, a catheter, or a catheter pump, preferably a stent, a stent graft, a graft, or a graft connector, and especially a stent or a stent graft.

It is to be especially preferred if the implant according to the present invention is coated with CA4 or CA4P.

A local administration of CA4 or CA4P is achieved by the direct release from the surface of a coronary or peripheral stent, which surface is coated with the active substance. To obtain a surface of a stent which is coated with the active substance, several technological approaches can be employed. Each of these approaches can be carried out to ensure that the active substance is released from the surface either within a short time (hours) or over an extended period of time (days). The release kinetics can be adjusted by specifically modifying the surface, e.g., by means of hydrophobic or hydrophilic side chains of a polymer substrate or a ceramic surface.

Ceramic Coating

An aluminum oxide coating (German Patent Applications No. DE 19855421 and No. DE 19910188 and International Publication No. WO 00/25841) with a porous surface can be coated with CA4 or CA4P in quantities between 5 and 500 μg either by immersion, spraying, or a comparable technique. The dose of the active substance depends on the type of target vessel and the condition of the patient and is selected to ensure that a proliferation or migration is sufficiently inhibited without inhibiting the healing process. CA4 or CA4P can be used in the form of an aqueous or organic solution, for example, in DMSO, DMF, ethanol, or methanol. After spraying or immersing the stent (under a weak vacuum), the treated stent is dried and this procedure is repeated 3-10 times. After the last drying step, the stent is rinsed for 1 min at room temperature in water or isotonic saline solution and subsequently dried. The content of active substance can be analyzed after lixiviating the active substance by means of a suitable solvent using standard methods (HPCL, LC-MS). The release kinetics can be measured using a standard release measuring device.

PTFE Membrane: Stent Graft

An approach comparable to the one described above is used. CA4 or CA4P is absorbed in the depressions of the porous PTFE membrane.

General Polymer Coating

A number of polymers are suitable for a coating with active substances: methacrylate polymers, polyurethane coatings, PTFE coatings. The active substance can be applied either onto the final surface (see above) or it can be added directly to the polymerization solution. As to the other details, this technical approach corresponds to the one already described above.

Mechanical Approach

The mechanical approach is based on holes which are cut into the braces of the stent by means of a slicing laser bean. These holes can subsequently be filled with CA4 or CA4P. The mechanical approach (using holes) can be combined with a thin biodegradable coating which itself contains the active substance. After an initial release from the biodegradable coating, the active substance can be released over a long period of time from the holes filled with the active substance. As to the other details, this technical approach corresponds to the one already described above.

Thus, another preferred embodiment of the implant according to the present invention provides that the implant have a ceramic coating, in particular one made of aluminum oxide, to which CA4 or CA4P is bonded.

According to yet another preferred embodiment of the implant according to the present invention, the implant is coated with a polymer coating, in particular one made of methacrylate polymers, polyurethane, or PTFE, especially PTFE, to which CA4 or CA4P is bonded or in which CA4 or CA4P was dissolved before the coating was applied.

According to another preferred embodiment of the implant according to the present invention, the metal of the implant has depressions which have been applied by means of a laser beam and which are filled with CA4 or CA4P. In this context, it particularly useful if the metal which has holes filled with CA4 or CA4P or at least the holes themselves is/are are coated with a biologically degradable polymer material, with CA4 or CA4P being optionally bonded to the polymer coating or with CA4 or CA4P being dissolved in the polymer material prior to polymerizing the coating.

According to another extremely useful embodiment of the implant according to the present invention, the implant can be manufactured using a method in which
a) an implant according to the present invention is used, which implant has a minimum of one closed or open-work layer or surface that consists of a metal or a metal alloy and that is homogeneous or formed from different strands (without, however, containing CA4 or CA4P) and which implant is coated with a ceramic coating, in particular aluminum oxide, or
b) an implant according to the present invention is used, which implant has a minimum of one closed or open-work layer or surface that consists of a polymer and that is homogeneous or formed from different strands (without, however, containing CA4 or CA4P), or
c) an implant according to the present invention (without, however, containing CA4 or CA4P) is used, which implant is coated with a polymerized coating or a coating which polymerizes on the surface, in particular methacrylate polymers, polyurethane, or PTFE, or
d) an implant according to the present invention (without, however, containing CA4 or CA4P) is used, with this implant having a minimum of one closed or open-work layer or surface which consists of a metal or a metal alloy, which is homogeneous or formed from different strands, and into which depressions have been made by means of a laser beam, which depressions are filled with CA4 or CA4P, and which implant is subsequently coated with a polymerized biodegradable coating or such a coating which polymerizes on the surface;
e) the implant is subsequently is brought into contact with a CA4 or CA4P solution in an aqueous or organic solvent according to a), b), c), or d), for example, by means of sprinkling, spraying, or immersion, possibly under a vacuum;
f) the implant is optionally dried, preferably until the solvent of step e) has been removed;
g) step e), optionally followed by step f), is repeated, preferably several times, in particular 3 to 10 times;
h) the implant subsequently is optionally rinsed once or several times with water or isotonic saline solution;
i) the implant optionally is subsequently dried.

It is to be preferred, if during the production of this implant according to the present invention, which implant can be produced as described above, CA4 or CA4P in step e) is dissolved in alcohol, preferably in ethanol or methanol, and/or if in step e), the implant, in vacuo, is brought into contact with a CA4 or CA4P solution in an aqueous or organic solvent by immersing it into said solution, preferably overnight, and/or if steps f) and/or g) are omitted and/or if in step h), the implant is repeatedly washed with saline solution and/or if in step i), the implant is dried overnight.

In an alternative and especially preferred embodiment of the present invention, it is especially useful if, during the production of the implant according to the present invention, which implant can be produced as described above, the implant in step e) is placed, preferably under aseptic conditions, into a preferably sterile container with a stopper which can be perforated and which, after the end of the perforation, seals itself, for example, into an injection bottle, if CA4 or CA4P solution, which is preferably sterile, is filled into the container, if this container is sealed with the stopper which can be perforated and which seals itself at the end of perforation, if a thin, preferably sterile, air-permeable air tube, for example, a cannula, is inserted into the stopper so as to perforate said stopper, if a vacuum is applied, and if the CA4 or CA4P solution is preferably stirred, and if finally, preferably after approximately 12 h, the thin, preferably sterile, air-permeable air tube is removed and/or if in step e), CA4 or CA4P is dissolved in alcohol, preferably in ethanol or methanol, and/or if the implant, until it is ready for use, remains in the preferably sterile, sealed glass container described in step e), and/or if steps f) through i) are omitted.

According to another highly advantageous embodiment of the implant according to the present invention, the implant can be produced using a method in which CA4 or CA4P is dissolved in the polymerization material prior to creating at least one closed or open-work polymer layer or surface or a polymer coating of the implant.

Furthermore, it is to be especially preferred if CA4 or CA4P is released after the implant according to the present invention has been implanted. It is to be especially preferred if the release takes place in a delayed form. According to an especially preferred embodiment of the present invention, CA4 or CA4P is released from the implant via a period of 24 h, preferably 48 h, and especially more than 96 h following implantation of the implant. In particular, it is useful if, after implantation, the CA4 or CA4P is released from the implant
a) within <48 h or
b) over a period of a minimum of 48 h, preferably over a minimum of 7 days, in particular over a minimum of 2 to 21 days, or if
c) the implant is able to release the active substance according to both release patterns a) and b).

The option mentioned last can be implemented by selecting two different types of coating, bonding, of physical fixation. As an example, the laser-produced holes with CA4 or CA4P which are sealed with biodegradable membranes that are coated with CA4 or CA4P. Following the rapid release [of the active substance] from the membrane, [the active substance] is released over a long period of time from the holes.

According to another preferred embodiment of the present invention, the implant contains a minimum of one additional active substance, preferably a pharmaceutical active substance, in particular an active substance chosen from the following group of active substances and their derivatives:
(Group 1): molsidomine, linsidomine, sodium nitroprusside, nitroglycerol, or NO donors in general; stimulators of the soluble guanylate cyclase (sGC), for example, BAY 41-2272 (5-(cyclopropyl-2-[1-(2-flurorobenzyl)-1H-pyrazolo[3,4-n]pyridin-3-yl]-pyrimidin-4-ylamine); hydralazine; verapamil, diltiazem, nefedipine, nimodipine, or Ca²⁺ channel blockers; captopril, enalapril, lisinopril, quinapril, or angiotensin converting enzyme inhibitors; losartan, candesartan, irbesartan, valsartan, or other antagonists of the angiotensin II receptor;
(Group 2): dexamethasone, betamethasone, prednisone, or other corticosteroids; 17-beta estradiol; cyclosporin; mycophenolic acid, VEGF, VEGF receptor activators; tranilast; meloxicam, Celebrex, Vioxx, or other COX-2 antagonists; indomethacin, diclofenac, ibuprofen, naproxen, or other COX-1 inhibitors; plasminogen activator inhibitors-1; thrombin inhibitors, for example, hirudin, Hirulog, argatroban, PPACK; or interleukin-10;
(Group 3): rapamycin, SDZ RAD (40-O-(2-hydroxyethyl)rapamycin or other rapamycin derivatives; FK506; PDGF antagonists; paclitaxel or 7-hexanoyltaxol; cisplatin; vinblastin; mitoxanthrone; topotecan; methotrexate; flavopiridol; actinomycin D; ReoPro/abciximab or probucol.

It is to be especially preferred if the additional active substance is one selected from group 1 and if this active substance is released from the implant within the first 24-72 h after implantation and/or if the additional active substance is one selected from group 2 and if this active substance is released from the implant within the first 48 h to 21 days after implantation and/or if the additional active substance is one selected from group 3 and if this active substance is released from the implant within 7 days to 3 months after implantation.

An additional subject matter of the present invention relates to a method for the production of an implant according to the present invention in which CA4 or CA4P is dissolved in the polymerization material prior to the creation of a minimum of one closed or open-work polymer layer or surface or a polymer coating of the implant.

An additional subject matter of the present invention also relates to a method for the production of an implant according to the present invention using the following steps:
a) an implant according to the present invention (without, however, containing CA4 or CA4P) which has a minimum of one closed or open-work layer or surface that consists of a metal or a metal alloy and that is homogeneous or formed from different strands, which implant is coated with a ceramic coating, in particular aluminum oxide, or
b) an implant according to the present invention (without, however, containing CA4 or CA4P) which has a minimum of one closed or open-work layer or surface that consists of a polymer and that is homogeneous or formed from different strands, or
c) an implant according to the present invention (without, however, containing CA4 or CA4P) which is coated with a polymerized coating or a coating which polymerizes on the surface, in particular methacrylate polymers, polyurethane, or PTFE, or
d) an implant according to the present invention (without, however, containing CA4 or CA4P), with this implant having a minimum of one closed or open-work layer or surface which consists of a metal or a metal alloy, which is homogeneous or formed from different strands, and into which depressions have been made by means of a laser beam, which depressions are filled with CA4 or CA4P, and with this implant subsequently being coated with a polymerized, preferably biodegradable coating or such a coating which polymerizes on the surface is used,
e) subsequently the implant is brought into contact with a CA4 or CA4P solution in an aqueous or organic solvent according to a), b), c), or d), for example, by means of sprinkling, spraying, or immersion, possibly under a vacuum,
f) subsequently the implant is optionally dried, preferably until the solvent of step e) has been removed,
g) subsequently step e), optionally followed by step f), is repeated, preferably several times, in particular 3 to 10 times, and
h) subsequently the implant is optionally rinsed once or several times with water or isotonic saline solution, and
i) subsequently the implant is optionally dried.

This method is to be especially preferred if in step e) CA4 or CA4P is dissolved in alcohol, preferably in ethanol or methanol, and/or if in step e) the implant, in vacuo, is brought into contact with a CA4 or CA4P solution in an aqueous or organic solvent by immersing it into said solution, preferably overnight, and/or if steps f) and/or g) are omitted and/or if in step h) the implant is repeatedly washed with saline solution and/or if in step i) the implant is dried overnight.

An especially preferred alternative of the method according to the present invention provides that in step e), the implant, preferably using aseptic means, be placed into a preferably sterile container with a stopper which can be perforated and which, after the end of the perforation, seals itself, for example, into an injection bottle, that CA4 or CA4P solution, which is preferably sterile, be filled into the container, that this container be closed with the stopper which can be perforated and which seals itself at the end of perforation, that a thin, preferably sterile, air-permeable air tube, for example, a cannula, be inserted into the stopper so as to perforate said stopper, that a vacuum be applied, and that the CA4 or CA4P solution be preferably agitated, and that finally, preferably after approximately 12 h, the thin, preferably sterile, air-permeable air tube be removed and/or that in step e), CA4 or CA4P be dissolved in alcohol, preferably in ethanol or methanol, and/or that the implant, until it is ready for use, remain in the preferably sterile, sealed glass container described in step e), and/or that steps f) through i) be omitted.

This particular method is especially favorable and completely unknown in prior art, and it offers considerable advantages in regard to both costs and production time and stops, especially since the implant is already packaged sterile as soon as it is produced.

An additional subject matter of the present invention relates to the use of an implant according to the present invention in the treatment or prophylaxis of vascular narrowings or stenoses, preferably in the prophylaxis of restenoses, in particular of coronary or peripheral vascular occlusions.

Another important subject matter of the present invention relates to the use of CA4 or CA4P, subsequently referred to as CA4 or CA4P administration, for coating or producing an implant to treat or prevent vascular narrowings or stenoses, preferably to prevent restenoses, in particular of coronary or peripheral vascular occlusions.

For the CA4 or CA4P administration, it is to be preferred if the implant is a stent, a stent graft, a graft, a graft connector, a guide wire, a catheter, or a catheter pump, preferably a stent, a stent graft, a graft, or a graft connector, and in particular a stent or a stent graft or a stent with a polymer surface.

Furthermore, for the CA4 or CA4P administration, it is to be preferred if the CA4 or CA4P is bonded or attached to the implant in such a way that it is released from the implant, preferably by delayed action, following implantation.

Yet another separate subject matter of this patent application refers to the use of CA4 or CA4P in the treatment or prophylaxis of vascular narrowings or stenoses, preferably in the prophylaxis of restenoses, in particular of coronary or peripheral vascular occlusions. As explained above and as discovered in the context of this patent application, to this end, CA4 or CA4P has especially favorable properties.

Still another subject matter of the present invention relates to the treatment of a patient or an animal requiring this treatment with or by means of an implant according to the present invention or a stent with a polymer surface.

Another subject matter of the present invention relates to the treatment of a patient or an animal requiring this treatment by means of a systemic administration of CA4 or CA4P. In this context, systemic refers to the oral, buccal, intravenous, subcutaneous, intramuscular, intratubular, pulmonary, rectal, intravaginal or nasal administration of the drug. The local administration by means of a suitable catheter is also feasible.

In the following section, the present invention will be explained in greater detail on the basis of examples, without, however, in any way limiting it to these examples.

### FIGURES AND EXAMPLES

Figures

FIG. 1 shows a graphic representation of two independent experiments which studied the inhibition of the SMC proliferation following the administration of CA4.

FIG. 2 shows a graphic representation of an experiment which studied the inhibition of the SMC proliferation following administration of paclitaxel.

### EXAMPLES

### Example 1

SMCs were plated out in a 96-well plate in a density of 2000 cells per well. After 24 h, different concentrations of the active substance were adjusted (10⁻² to 10⁻¹⁰M). The proliferation was measured after 24, 48, 72, and 96 h following the addition of the active substance using a BrdU-ELISA or, in the case of paclitaxel, by counting the number of cells.

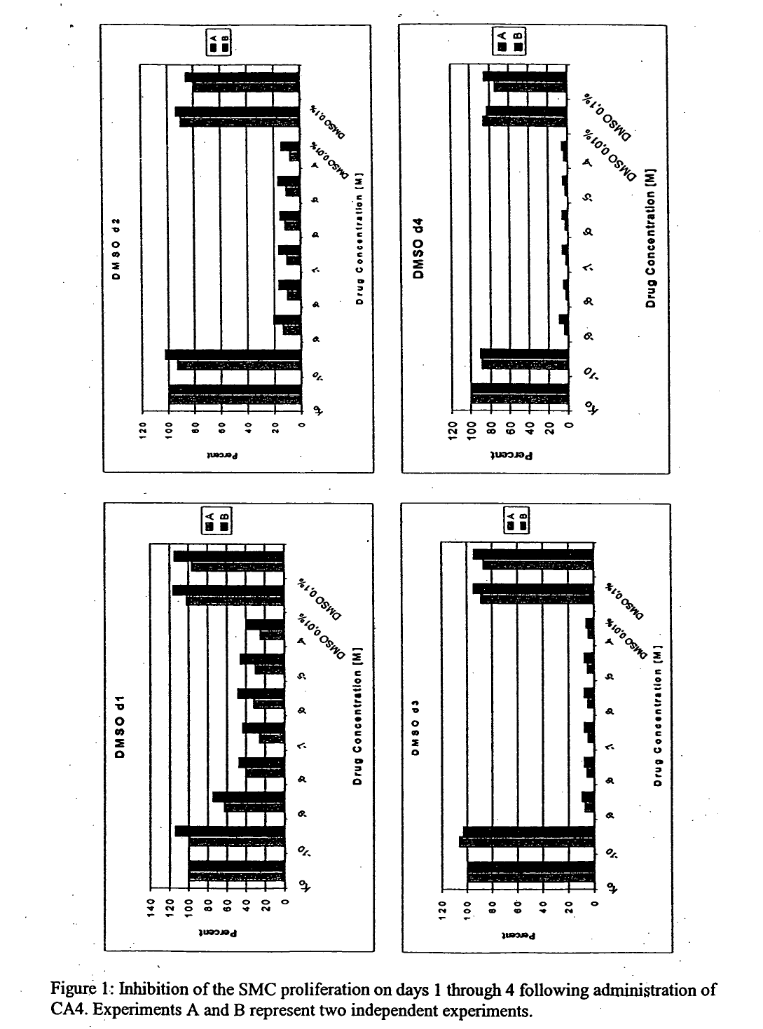

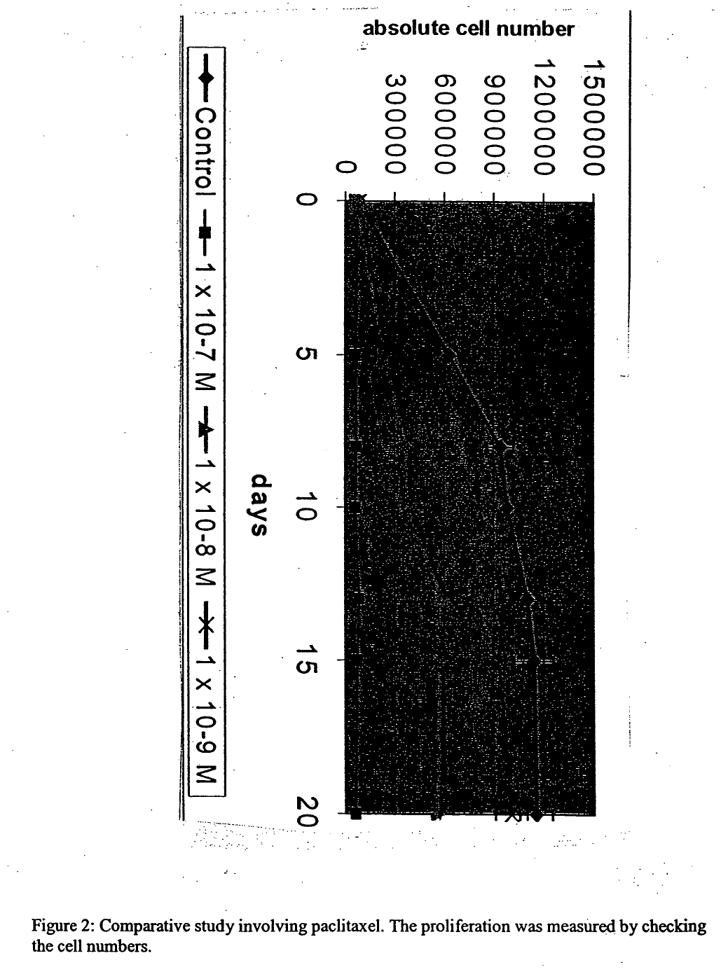

The results of the experiments are summarized in FIGS. 1 and 2 and show that in the range from 5×10⁻⁹ to 10⁻¹⁰M, the cell growth had been dose-dependently inhibited. This shows that CA4 has an IC₅₀ of 0.5 nM while paclitaxel has an IC₅₀ of approximately 10 nM. Thus, CA4 is 10 to 50 times more effective than paclitaxel.

### Example 2

Method (1) of manufacturing especially stents coated with CA4 or CA4P:

10 mg of CA4 or CA4P are dissolved in 3 mL of ethanol or water.

The implants are immersed overnight in the solution in vacuo and at room temperature.

They are washed three times for 1 min with saline solution.

They are dried overnight.

### Example 3

New alternative method (2) of manufacturing in particular sterile stents and stent grafts

Small injection bottles that are not much larger than the stent are used.

Sterile coronary stent grafts (CSGs) are placed into the sterile injection bottles under aseptic conditions.

0.5 mL of the aseptically filtered CA4 or CA4P solution (3.3 mg/ml in ethanol or water) is added to the bottle.

The bottles are sealed with a rubber stopper.

A sterile injection cannula with an aseptic filter is pushed through the center of the rubber stopper.

The bottles are horizontally placed under a vacuum on a roller apparatus into a desiccator.

The bottles are rolled overnight under a vacuum.

The injection needle is removed.

No rinsing takes place.

The sterile CSGs are ready for use.

### Example 4

Another example is a stent which releases the active substance, especially one with a plurality of layers, for example, stents with a polymer surface and stent grafts, etc., which, in addition to CA4 or CA4P, contain a minimum of one, two, three, or more additional active substances and correspondingly release them. A selection of possible active substances is listed below. The active substances listed also comprise the corresponding derivatives and all types of salts, enantiomers, racemates, bases, or free acids.

Depending on their preferred release profile or release time, the active substances listed are divided into groups 1 to 3. It is to be preferred if the stents or stent grafts contain active substance from different groups.

The loading methods will be described below under the technical approaches.

Phase I—Vasodilation (Group 1)

Active substances which are released especially within the first 24-72 h after stent placement.

Active substance

Molsidomine, linsidomine, sodium nitroprusside, nitroglycerol, or NO donors in general

Stimulators of the soluble guanylate cyclase, such as BAY 41-2272 (5-(cyclopropyl-2-[1-(2-flurorobenzyl)-1H-pyrazolo[3,4-n]pyridin-3-yl]-pyrimidin-4-ylamine)Hydralazine

Verapamil, diltiazem, nefedipine, nimodipine, and other Ca²⁺ channel blockers

Captopril, enalapril, lisinopril, quinapril, and other angiotensin converting enzyme inhibitors

Losartan, candesartan, irbesartan, valsartan, and other antagonists of the angiotensin II receptor

Phase II—Inhibition of Inflammations, Immunosuppression, Promotion of the Cell Growth of Endothelial Cells, Inhibition of the Cell Migration (Group 2)

Active substances which are released especially within the first 2-21 days after stent placement.

Active substance

Dexamethasone, betamethasone, prednisone, and other corticosteroids

17-beta estradiol

Cyclosporin

Mycophenolic acid

VEGF, VEGF receptor activators

Tranilast

Meloxicam, Celebrex, Vioxx, and other COX-2 antagonists

Indomethacin, diclofenac, ibuprofen, naproxen, and other COX-1 inhibitors

Plasminogen activator inhibitors-1 and other serpines

Thrombin inhibitors, such as hirudin, Hirulog, argatroban,.PPACK, etc.

Interleukin-10

Phase III—Inhibition of the Cell Proliferation (Group 3)

Active substances which are released especially within the first 14 days to 3 months following stent placement.

Active substance

SDZ RAD (40-O-(2-hydroxyethyl)rapamycin or other rapamycin derivatives

PDGF antagonists

FK506 (tacrolimus)

Paclitaxel

Cisplatin

Vinblastin

Mitoxanthrone

Topotecan

Methotrexate

Flavopiridol

A local administration of the active substance is achieved by the direct release from the surface of a coronary or peripheral stent, which surface is coated with the active substance. To obtain a surface of a stent which is coated with the active substance, several technological approaches can be employed. Each of these approaches can be carried out to ensure that the active substance is released from the surface either within a short time (hours) or over an extended period of time (days). The release kinetics can be adjusted by specifically modifying the surface, e.g., by means of hydrophobic or hydrophilic side chains of a polymer substrate or a ceramic surface.

Ceramic Coating

An aluminum oxide coating (German Patent Applications No. DE 19855421 and No. DE 19910188 and International Publication No. WO 00/25841) with a porous surface can be coated with the active substance (for example, CA4 or CA4P in quantities between 5 and 500 μg) either by immersion, spraying, or a comparable technique. The dose of the active substance depends on the type of target vessel and the condition of the patient and is selected to ensure that a proliferation, migration, or T-cell response is sufficiently inhibited without inhibiting the healing process. The active substance can be used in the form of an aqueous or organic solution, for example, in DMSO, DMF, ethanol, or methanol. After spraying or immersing the stent (under a weak vacuum), the treated stent is dried and this procedure is repeated 3-10 times. After the last drying step, the stent is rinsed for 1 min at room temperature in water or isotonic saline solution and subsequently dried. The content of active substance can be analyzed after lixiviating the active substance by means of a suitable solvent using standard methods (HPCL, LC-MS). The release kinetics can be measured using a standard release measuring device.

PTFE Membrane: Stent Graft

An approach comparable to the one described above is used. The active substance is absorbed in the depressions of the porous PTFE membrane.

General Polymer Coating

A number of polymers are suitable for a coating with active substances: methacrylate polymers, polyurethane coatings, PTFE coatings. The active substance can be applied either onto the end surface (see above) or it can be added directly to the polymerization solution. As to the other details, this technical approach corresponds to the one already described above.

Mechanical Approach

The mechanical approach is based on holes which are cut into the braces of the stent by means of a slicing laser beam. These holes can subsequently be filled with the active substance. The mechanical approach (using holes) can be combined with a thin biodegradable coating which itself contains the active substance. After an initial release from the biodegradable coating, the active substance can be released over a long period of time from the holes filled with the active substance. As to the other details, this technical approach corresponds to the one already described above.

1-34. (cancelled)

35. An implant containing CA4 or CA4P in a covalently or noncovalently chemical bonded or physically fixed form.

36. The implant of claim 35, wherein said implant is an intracavernous implant.

37. The implant of claim 35, wherein said implant is an intravascular implant.

38. The implant of claim 35, wherein said implant has at least one closed or open-work layer or surface which is homogeneous or formed from different strands.

39. The implant of claim 38, wherein a surface of said implant comprises a polymer.

40. The implant of claim 38, wherein a surface of said implant comprises a metal or metal alloy.

41. The implant of claim 35, further comprising at least one polymer layer that completely or partially covers a closed or open-work layer or surface that is homogeneous or formed from different strands.

42. The implant of claim 41, wherein said closed or open-work layer or surface comprises a metal or a metal alloy.

43. The implant of claim 42, wherein said open-work layer or surface is lattice-like.

44. The implant of claim 35, wherein said implant has at least one closed or open-work first layer or surface comprising a metal or a metal alloy, and wherein said first layer or surface is homogeneous or formed from different strands; and at least one closed or open-work second layer or surface comprising a polymer and wherein said first layer or surface is homogeneous or formed from different strands.

45. The implant of claim 44, wherein said first layer(s) is a lattice-like structure and wherein said second layer(s) is homogeneously closed or woven, and impermeable to water or corpuscles.

46. The implant of claim 45, wherein said layers are arranged in a sequence, from the outside to the inside of the implant, selected from the group consisting of:
a) metal-polymer;
b) polymer-metal;
c) metal-polymer-metal; and
d) polymer-metal-polymer.

47. The implant of claim 46, wherein said second (polymer) layer is not chemically bonded to said first (metal or metal alloy) layer.

48. The implant of claim 46, wherein said second (polymer) layer is bonded by an adhesive to said first (metal or metal alloy) layer.

49. The implant of claim 39, wherein said polymer is selected from the group consisting of Dacron; expandable polytetrafluoroethylene (PTFE/Teflon®), unexpandable polytetrafluoroethylene (PTFE/Teflon®), and polyurethane.

50. The implant of claim 35, wherein said implant is a stent, a stent graft, a graft, a graft connector, a guide wire, a catheter, or a catheter pump.

51. The implant of claim 35, wherein the CA4 or CA4P is coated thereon.

52. The implant of claim 35, wherein said implant has a ceramic coating to which CA4 or CA4P is bonded.

53. The implant of claim 52, wherein said ceramic coating comprises aluminum oxide.

54. The implant of claim 35, wherein said implant has a polymer coating to which CA4 or CA4P is bonded or incorporated therein.

55. The implant of claim 35, wherein the polymer coating is selected from the group consisting of methacrylate polymers, polyurethane, and PTFE.

56. The implant of claim 40, wherein said metal or metal alloy has depressions which have been applied by a laser beam, and into which depressions are deposited CA4 or CA4P.

57. The implant of claim 56, wherein said metal or metal alloy has holes filled with CA4 or CA4P.

58. The implant of claim 56, wherein said metal or metal alloy has holes coated with a biologically degradable polymer material.

59. The implant of claim 58, wherein said CA4 or CA4P are bonded to said biologically degradable polymer or dissolved said biologically degradable polymer material prior to polymerizing said biologically degradable polymer.

60. A method of manufacturing an implant of claim 35, comprising a contact step in which one of the following implants is subsequently brought into contact with a CA4 or CA4P solution in an aqueous or organic solvent:
a) an implant having at least one closed or open-work layer or surface comprising a metal or a metal alloy and that is homogeneous or formed from different strands, and wherein said implant is coated with a ceramic coating;
b) an implant having at least one closed or open-work layer or surface comprising a polymer and that is homogeneous or formed from different strands;
c) an implant coated with a polymerized coating or a coating which polymerizes on the surface, or
d) an implant having at least one closed or open-work layer or surface comprising a metal or a metal alloy which is homogeneous or formed from different strands, and into which depressions have been made by a laser beam, and wherein said depressions are filled with CA4 or CA4P, and wherein said implant is subsequently coated with a polymerized biodegradable coating or such a coating which polymerizes on the surface.

61. The method of claim 60, wherein said contact with a CA4 or CA4P solution in an aqueous or organic solvent is followed by a drying step to remove said solvent.

62. The method of claim 60, wherein said contact step is repeated one or more times.

63. The method of claim 62, wherein each contact step is followed by a drying step to remove said solvent.

64. The method of claim 62, wherein each contact step is repeated 3 to 10 times.

65. The method of claim 62, wherein each contact step is followed by a rinsing step, wherein said implant is subsequently contacted one or more times with water or isotonic saline solution.

66. The method of claim 65, wherein each rinsing step is followed by a drying step to remove the solvent.

67. The method of claim 60, wherein said CA4 or CA4P is dissolved in alcohol to form said CA4 or CA4P solution, preferably in ethanol or methanol to form the CA4 or CA4P solution.

68. The method of claim 60, wherein said CA4 or CA4P is dissolved in alcohol to form said CA4 or CA4P solution, preferably in ethanol or methanol to form the CA4 or CA4P solution.

69. The method of claim 68, wherein said alcohol is ethanol or methanol.

70. The method of claim 60, wherein said implant is brought into contact with said CA4 or CA4P solution in an aqueous or organic solvent by immersion into said solution,

71. The method of claim 70, wherein said immersion is done overnight.

72. The method of claim 70, wherein said implant is subsequently washed repeatedly with saline solution.

73. A method of manufacturing an implant of claim 35 with at least one closed or open-work polymer layer or surface which is homogeneous or formed from different strands, wherein CA4 or CA4P is dissolved in the polymerization material prior to the creation of polymer layer or surface.

74. The implant of claim 35, wherein said CA4 or CA4P is released after the implant has been implanted.

75. The implant of claim 74, wherein said release is by delayed action.

76. The implant of claim 75, wherein said CA4 or CA4P is released from said implant over a period of about 24-96 h following implantation of the implant.

77. The implant of claim 76, wherein said CA4 or CA4P is released from the implant
i) within <48 h;
ii) over a period of from at least 2 to 21 days, or that
iii) the implant is able to release the active substance according to both release patterns i) and ii).

78. The implant of claim 35, wherein said implant contains at least one additional pharmaceutically active substance.

79. The implant of claim 78, wherein said pharmaceutically active substance is selected from the group consisting of:
i) molsidomine, linsidomine, sodium nitroprusside, nitroglycerol, or other NO donors;
ii) BAY 41-2272 (5-(cyclopropyl-2-[1-(2-flurorobenzyl)-1H-pyrazolo [3,4-n]pyridin-3-yl]-pyrimidin-4-ylamine) other stimulators of the soluble guanylate cyclase (sGC);
iii) hydralazine;
iv) verapamil, diltiazem, nefedipine, nimodipine, or other Ca²⁺ channel blockers;
v) captopril, enalapril, lisinopril, quinapril, or other angiotensin converting enzyme inhibitors; and
vi) losartan, candesartan, irbesartan, valsartan, or other antagonists of the angiotensin 11 receptor.

80. The implant of claim 79, wherein said pharmaceutically active substance is released within the first 24-72 h after implantation.

81. The implant of claim 78, wherein said pharmaceutically active substance is selected from the group consisting of:
i) dexamethasone, betamethasone, prednisone, or other corticosteroids;
ii) 17-beta estradiol;
iii) cyclosporin;
iv) mycophenolic acid;
v) VEGF or VEGF receptor activators;
vi) tranilast;
vii) meloxicam, Celebrex, Vioxx, or other COX-2 antagonists;
viii) indomethacin, diclofenac, ibuprofen, naproxen, or other COX-1 inhibitors;
ix) plasminogen activator inhibitors-1;
x) hirudin, Hirulog, argatroban, PPACK or other thrombin inhibitors; and
xi) interleukin-10.

82. The implant of claim 81, wherein said pharmaceutically active substance is released from the implant within the first 48 hours to 21 days after implantation.

83. The implant of claim 78, wherein said pharmaceutically active substance is selected from the group consisting of:
i) rapamycin, SDZ RAD (40-O-(2-hydroxyethyl) rapamycin or other rapamycin derivatives;
ii) FK506;
iii) PDGF antagonists; and
iv) paclitaxel, 7-hexanoyltaxol, cisplatin; vinblastine, mitoxanthrone, topotecan, methotrexate, flavopiridol, actinomycin D, ReoPro/abciximab, probucol and other chemotherapeutic agents.

84. The implant of claim 83, wherein said pharmaceutically active substance is released from the implant within 7 days to 3 months after implantation.

85. A method of using an implant containing CA4 or CA4P in a chemically covalently or noncovalently bonded or physically fixed form for the treatment or prophylaxis of vessel narrowings or stenoses, comprising introducing said implant into a patient.

86. The method of claim 85, wherein said vessel narrowings or stenoses are formed by restenosis.

87. The method of claim 85, wherein said vessel narrowing or stenoses form coronary or peripheral vascular occlusions.

88. The method of claim 85, wherein said CA4 or CA4P is bonded or attached to the implant in such a way that it is released from the implant following implantation.

89. The method of claim 85, wherein said the implant is selected from the group consisting of a stent, a stent graft, a graft, a graft connector, a guide wire, a catheter, or a catheter pump.
